(19) United States
(12) Patent Application Publication
(10) Pub. No.: US 2023/0065296 A1
(43) Pub. Date: Mar. 2, 2023

(54) EYE-TRACKING USING EMBEDDED ELECTRODES IN A WEARABLE DEVICE

(71) Applicant: Facebook Technologies, LLC, Menlo Park, CA (US)

(72) Inventors: Morteza Khaleghimeybodi, Bothell, WA (US); Nava K. Balsam, Woodinville, WA (US); Nils Thomas Fritiof Lunner, Redmond, WA (US)

(21) Appl. No.: 17/461,769

(22) Filed: Aug. 30, 2021

(51) Int. Cl. A61B 5/16; A61B 5/00; A61B 5/24

(52) U.S. Cl. CPC A61B 5/163; A61B 5/6803; A61B 5/7225; A61B 5/7267; A61B 5/6817; A61B 5/2415; A61B 2562/0204; A61B 2562/0215

(57) ABSTRACT

A wearable device assembly is described for determining eye-tracking information of a user using generated biopotential signals on the head of the user. The eye tracking system monitors biopotential signals generated on a head of a user using electrodes that are mounted on a device that is coupled to the head of the user. The system determines eye-tracking information for the user based on the monitored biopotential signals using a machine learning model. The system performs at least one action based in part on the determined eye-tracking information.

### FIELD OF THE INVENTION

This disclosure relates generally to an eye-tracking system in a headset, and specifically relates to enhancing eye tracking using biopotential signals derived from embedded electrodes in the headset.

### BACKGROUND

Headsets often include features such as eye-tracking sensors to provide enhanced visual or audio content experience to users of the headsets. Typically, the eye-tracking is performed by camera-based eye-tracking sensors that track eye-ball movement by capturing corneal reflections at different gaze positions. Accordingly, conventional systems may not perform eye-tracking at a desired level of accuracy when it is difficult to capture the corneal reflections in certain situations. For example, when an eye is occluded or when a level of ambient light is low, there may be poor information from the corneal reflections. Furthermore, when a power level of the eye-tracking system is low, it may not be feasible to use the camera-based eye-tracking sensors. Such issues may lead to a reduced level of performance in the eye-tracking performed by the camera-based eye-tracking sensors.

### SUMMARY

An eye-tracking system is described herein that monitors electrophysiological signals from a plurality of electrodes to determine information associated with eye movements of the user. The system may be a hybrid system that optionally includes information from one or more eye-tracking cameras. The eye-tracking system is part of a head mounted system (e.g., headset and/or in-ear devices) that may provide eye-tracking information of a user wearing the head mounted system. The eye-tracking system may measure the electrophysiological signals (also termed biopotential signals) using an electrode assembly that includes a plurality of electrooculography (EOG) electrodes. The eye-tracking system determines eye-tracking information based on the measured biopotential signals using a trained machine learning model. In some embodiments, the information from the eye-tracking system may be used to identify gaze information and perform actions such as selectively emphasizing acoustic content that is received from particular acoustic sensors in the head mounted system, adjusting the display of virtual content at a display in the head mounted system, inferring the direction of arrival (DOA) estimation and steering the beamforming algorithm towards that direction so the audio capture is enhanced selectively in that direction, etc.

In embodiments described herein, the system monitors biopotential signals received from a plurality of electrodes mounted on a device that is coupled to a head of a user. The system determines eye-tracking information for the user using a trained machine learning model based on the monitored biopotential signals. The system performs at least one action based in part on the determined eye-tracking information.

In some embodiments a wearable device assembly is described. The wearable device assembly includes a headset. The headset includes a display assembly, an audio system, and an eye-tracking system. The eye tracking system is configured to receive biopotential signals from a plurality of electrodes that are configured to monitor biopotential signals generated within a head of a user in response to eye movements of the user. The eye tracking system also determines eye-tracking information for the user using a trained machine learning model based on the monitored biopotential signals. At least one of the display assemblies and the audio system are configured to perform at least one action based in part on the determined eye-tracking information.

### BRIEF DESCRIPTION OF THE DRAWINGS

FIG. 1A is a perspective view of a headset implemented as an eyewear device, in accordance with one or more embodiments.

FIG. 1B is a perspective view of a headset implemented as a head-mounted display, in accordance with one or more embodiments.

FIG. 2 is a profile view of a portion of an in-ear device, in accordance with one or more embodiments

FIG. 3 is a cross section/side view of a headset with electrodes displayed relative to a user's eye, in accordance with one or more embodiments.

FIG. 4A is a block diagram of a wearable device assembly with an optional in-ear device, in accordance with one or more embodiments.

FIG. 4B is a block diagram of an audio system, in accordance with one or more embodiments.

FIG. 4C is a block diagram of an eye-tracking system, in accordance with one or more embodiments.

FIG. 5 is a flowchart illustrating a process for determining and using eye-tracking information from monitored biopotential signals, in accordance with one or more embodiments.

FIG. 6 is a block diagram of a system environment that includes a headset with an eye tracking system, an optional in-ear device assembly, and a console, in accordance with one or more embodiments.

The figures depict various embodiments for purposes of illustration only. One skilled in the art will readily recognize from the following discussion that alternative embodiments of the structures and methods illustrated herein may be employed without departing from the principles described herein.

### DETAILED DESCRIPTION

The present disclosure generally relates to determining eye-tracking information, and specifically relates to monitoring and using biopotential signals generated on a head of a user using EOG electrodes. The monitored biopotential signals may be optionally combined with eye tracking information received from eye tracking sensors in a headset.

Eye tracking refers to the process of detecting the direction of a user's gaze, which may detect angular orientation of the eye in 3-dimensional (3D) space. Additionally, eye tracking may detect a location of the eye (e.g., the center of the eye), a torsion (i.e., the roll of the eye about the pupillary axis) of the eye, a shape of the eye, a current focal distance of the eye, a dilation of the pupil, or other features of the eye's state. One conventional technique for eye tracking captures video images of a user and identifies an orientation of the user's pupils using a machine vision algorithm. However, this technique consumes substantial computing resources, and is susceptible to occlusion of the eye by eyelashes and eyelids. Furthermore, this method is affected by contrast between the iris and the pupil, which may vary for different users. Thus, video-based pupil tracking may not be able to accurately track the eyes of a user with dark irises. Capturing video images of a user to determine the direction of the user's gaze in a virtual reality headset has additional drawbacks. For example, types of cameras for capturing images from which an orientation of a user's pupil may be determined are typically relatively expensive or large. In addition, camera-based (e.g., imaging-based) eye-tracking techniques capture information at the framerate speed of the camera. In most cases, the framerate of the camera is relatively slow (<60 Hz). This relatively slow capture rate may pose some constraints in capturing rapid eye movements (e.g., saccadic movements). Such techniques may also place constraints on the proximity of the camera to the user's eye, which places constraints on the device used for eye-tracking. However, when performing eye-tracking in a virtual reality environment, using a detection element that is small and relatively close to the user's eye for eye tracking may be preferred. Additionally, video-based eye-tracking cannot track orientation of a user's eye while the user's eye is closed (e.g., when the user is blinking).

An eye-tracking system is described herein that monitors biopotential signals information from a plurality of EOG electrodes to determine information associated with eye movements of the user. The system may be a hybrid system that optionally includes information from one or more eye-tracking cameras. In such a hybrid system, the information from a camera-based eye-tracking system is combined with information from the biopotential-based eye tracking system to realize a multi-modal hybrid eye-tracking system. The multi-modal hybrid eye-tracking system may improve tracking in corner cases such as where the eyelids are covering the eyeballs. The eye-tracking system is part of a head mounted system (e.g., headset and/or in-ear devices) that may provide eye-tracking information of a user wearing the head mounted system. The eye-tracking system may measure the biopotential signals using an electrode assembly including a plurality of EOG electrodes. In some embodiments, the electrode assembly may be embedded in the head mounted system. For example, the electrode assembly may be part of one or both of a headset and/or one or more in-ear devices. In some embodiments, the eye-tracking system may combine the eye-tracking information received from the electrode assembly together with information received from eye-tracking cameras on the headset. The eye-tracking system determine eye-tracking information based on the measured biopotential signals using a trained machine learning model. In some embodiments, the information from the eye-tracking system may be perform selective actions such as selectively emphasizing acoustic content that is received from particular acoustic sensors in the head mounted system, adjusting the display of virtual content at a display in the head mounted system, etc.

While conventionally one or more eye-tracking cameras may be used to determine information associated with eye-movements of the user, there are advantages to instead using an electrode assembly with a plurality of EOG electrodes within a head mounted system and monitoring the biopotential signals generated at the plurality of EOG electrodes. One advantage is that the power requirements of the electrode assembly are much lower than the power requirements of the eye-tracking cameras. Thus, in situations where the head mounted system may be experiencing low power, the electrode assembly may continue to monitor the biopotential signals generated due to eye movements of the user, while any eye-tracking cameras may provide poor information due to the low power situation. Another advantage is that the biopotential signals monitored by the electrode assembly are not affected by occlusion effects such as may occur during eye blinks. In contrast, the eye-tracking cameras may obtain incorrect eye-tracking information during eye-blinks. Furthermore, the biopotential signals monitored by the EOG electrodes are obtained at a higher sampling frequency than a sampling frequency used to track eye movements by the eye-tracking cameras. As a consequence of this higher sampling frequency, the eye-tracking information received from the electrode assembly may lead to more uninterrupted eye-tracking that the information received from the eye-tracking cameras alone.

Embodiments of the invention may include or be implemented in conjunction with an artificial reality system. Artificial reality is a form of reality that has been adjusted in some manner before presentation to a user, which may include, e.g., a virtual reality (VR), an augmented reality (AR), a mixed reality (MR), a hybrid reality, or some combination and/or derivatives thereof. Artificial reality content may include completely generated content or generated content combined with captured (e.g., real-world) content. The artificial reality content may include video, audio, haptic feedback, or some combination thereof, any of which may be presented in a single channel or in multiple channels (such as stereo video that produces a three-dimensional effect to the viewer). Additionally, in some embodiments, artificial reality may also be associated with applications, products, accessories, services, or some combination thereof, that are used to create content in an artificial reality and/or are otherwise used in an artificial reality. The artificial reality system that provides the artificial reality content may be implemented on various platforms, including a wearable device (e.g., headset) connected to a host computer system, a standalone wearable device (e.g., headset), a mobile device or computing system, or any other hardware platform capable of providing artificial reality content to one or more viewers.

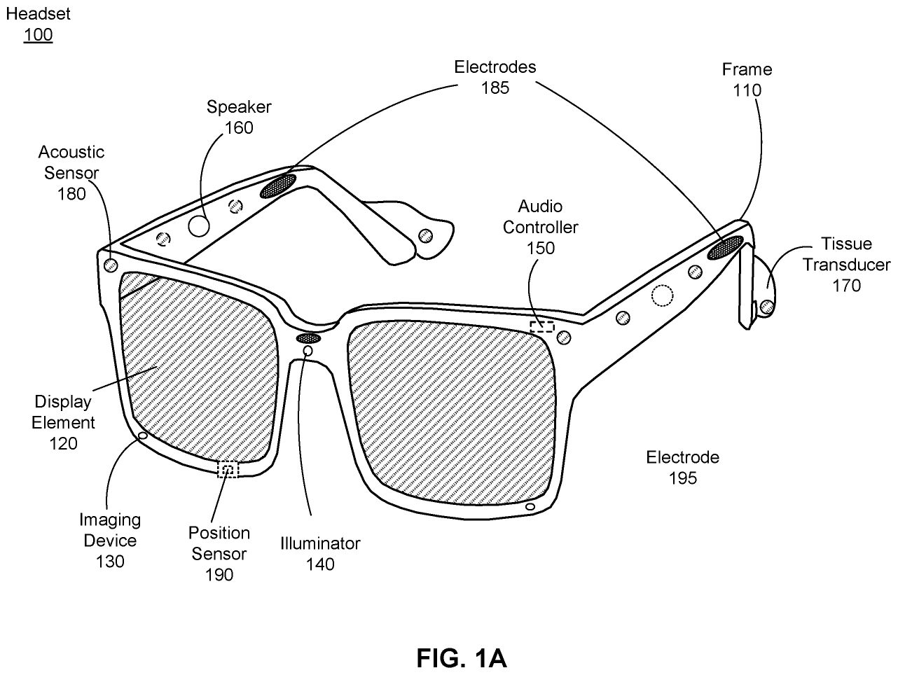

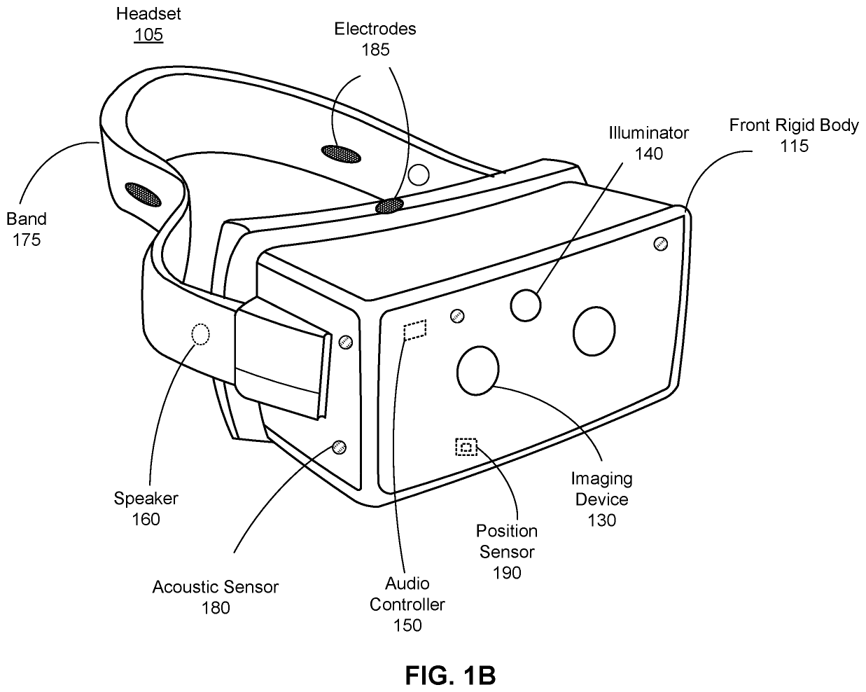

FIG. 1A is a perspective view of a headset 100 implemented as an eyewear device, in accordance with one or more embodiments. In some embodiments, the eyewear device is a near eye display (NED). In some embodiments, the headset 100 may be a client device. In general, the headset 100 may be worn on the face of a user such that content (e.g., media content) is presented using a display assembly and/or an audio system. However, the headset 100 may also be used such that media content is presented to a user in a different manner. Examples of media content presented by the headset 100 include one or more images, video, audio, or some combination thereof. The headset 100 includes a frame, and may include, among other components, a display assembly including one or more display elements 120, a depth camera assembly (DCA), an audio system, and a position sensor 190. While FIG. 1A illustrates the components of the headset 100 in example locations on the headset 100, the components may be located elsewhere on the headset 100, on a peripheral device paired with the headset 100, or some combination thereof. Similarly, there may be more or fewer components on the headset 100 than what is shown in FIG. 1A.

The frame 110 holds the other components of the headset 100. The frame 110 includes a front part that holds the one or more display elements 120 and end pieces (e.g., temples) to attach to a head of the user. The front part of the frame 110 bridges the top of a nose of the user. The length of the end pieces may be adjustable (e.g., adjustable temple length) to fit different users. The end pieces may also include a portion that curls behind the ear of the user (e.g., temple tip, ear piece).

The one or more display elements 120 provide light to a user wearing the headset 100. As illustrated the headset includes a display element 120 for each eye of a user. In some embodiments, a display element 120 generates image light that is provided to an eyebox of the headset 100. The eyebox is a location in space that an eye of user occupies while wearing the headset 100. For example, a display element 120 may be a waveguide display. A waveguide display includes a light source (e.g., a two-dimensional source, one or more line sources, one or more point sources, etc.) and one or more waveguides. Light from the light source is in-coupled into the one or more waveguides which outputs the light in a manner such that there is pupil replication in an eyebox of the headset 100. In-coupling and/or outcoupling of light from the one or more waveguides may be done using one or more diffraction gratings. In some embodiments, the waveguide display includes a scanning element (e.g., waveguide, mirror, etc.) that scans light from the light source as it is in-coupled into the one or more waveguides. Note that in some embodiments, one or both of the display elements 120 are opaque and do not transmit light from a local area around the headset 100. The local area is the area surrounding the headset 100. For example, the local area may be a room that a user wearing the headset 100 is inside, or the user wearing the headset 100 may be outside and the local area is an outside area. In this context, the headset 100 generates VR content. Alternatively, in some embodiments, one or both of the display elements 120 are at least partially transparent, such that light from the local area may be combined with light from the one or more display elements to produce AR and/or MR content.

In some embodiments, a display element 120 does not generate image light, and instead is a lens that transmits light from the local area to the eyebox. For example, one or both of the display elements 120 may be a lens without correction (non-prescription) or a prescription lens (e.g., single vision, bifocal and trifocal, or progressive) to help correct for defects in a user's eyesight. In some embodiments, the display element 120 may be polarized and/or tinted to protect the user's eyes from the sun.

In some embodiments, the display element 120 may include an additional optics block (not shown). The optics block may include one or more optical elements (e.g., lens, Fresnel lens, etc.) that direct light from the display element 120 to the eyebox. The optics block may, e.g., correct for aberrations in some or all of the image content, magnify some or all of the image, or some combination thereof.

In some embodiments, the display element 120 may receive eye-tracking information from an eye-tracking system (not shown). The received eye-tracking information may include a determination of occurrence of one or more ocular events. The display element 120 may adjust the display of visual content presented to the user based on the information associated with the determined one or more ocular events.

The DCA determines depth information for a portion of a local area surrounding the headset 100. The DCA includes one or more imaging devices 130 and a DCA controller (not shown in FIG. 1A), and may also include an illuminator 140. In some embodiments, the illuminator 140 illuminates a portion of the local area with light. The light may be, e.g., structured light (e.g., dot pattern, bars, etc.) in the infrared (IR), IR flash for time-of-flight, etc. In some embodiments, the one or more imaging devices 130 capture images of the portion of the local area that include the light from the illuminator 140. As illustrated, FIG. 1A shows a single illuminator 140 and two imaging devices 130. In alternate embodiments, there is no illuminator 140 and at least two imaging devices 130.

The DCA controller computes depth information for the portion of the local area using the captured images and one or more depth determination techniques. The depth determination technique may be, e.g., direct time-of-flight (ToF) depth sensing, indirect ToF depth sensing, structured light, passive stereo analysis, active stereo analysis (uses texture added to the scene by light from the illuminator 140), some other technique to determine depth of a scene, or some combination thereof.

The DCA may include an eye tracking unit that determines eye-tracking information. The eye-tracking information may comprise information about a position and an orientation of one or both eyes (within their respective eye-boxes). In some embodiments, the eye-tracking unit may include one or more eye-tracking cameras (not shown) that detect corneal reflections at different gaze positions from the eye of the user of the headset 100. The eye-tracking unit estimates an angular orientation of one or both eyes based on images captured of one or both eyes by the one or more cameras. In some embodiments, the eye-tracking unit may also include one or more illuminators that illuminate one or both eyes with an illumination pattern (e.g., structured light, glints, etc.). The eye-tracking unit may use the illumination pattern in the captured images to determine the eye-tracking information. The headset 100 may prompt the user to opt in to allow operation of the eye-tracking unit. For example, by opting in the headset 100 may detect, store, images of the user's any or eye-tracking information of the user.

In some embodiments, the eye-tracking unit includes a plurality of electrodes 185 that form an electrode assembly. The electrodes 185 monitor biopotential signals generated within a head of the user in response to the occurrence of ocular events such as eye movements, saccades, eye blinks, etc. The electrodes 185 are coupled to and/or attached to different portions of the head mounted system and are in direct contact with the skin on the head of the user. The electrodes 185 are part of an eye-tracking system that provides eye-tracking information to other systems in the headset 100. As illustrated, the electrodes 185 are located on the frame, at the nose bridge as well as the end pieces of the frame but in other embodiments, the electrodes 185 may be located on other portions of the head mounted system and portions of in-ear devices, portions of hearing aids, portions of hearables, or some combination thereof.

The audio system provides audio content. The audio system includes a transducer array, a sensor array, and an audio controller 150. However, in other embodiments, the audio system may include different and/or additional components. Similarly, in some cases, functionality described with reference to the components of the audio system can be distributed among the components in a different manner than is described here. For example, some or all of the functions of the controller may be performed by a remote server.

The transducer array presents audio content to the user. The transducer array includes a plurality of transducers. A transducer may be a speaker 160 or a tissue transducer 170 (e.g., a bone conduction transducer or a cartilage conduction transducer). Although the speakers 160 are shown exterior to the frame 110, the speakers 160 may be enclosed in the frame 110. In some embodiments, instead of individual speakers for each ear, the headset 100 includes a speaker array comprising multiple speakers integrated into the frame 110 to improve directionality of presented audio content. The tissue transducer 170 couples to the head of the user and directly vibrates tissue (e.g., bone or cartilage) of the user to generate audio signals. The number and/or locations of transducers may be different from what is shown in FIG. 1A.

The sensor array detects sounds within the local area of the headset 100. The sensor array includes a plurality of acoustic sensors 180. An acoustic sensor 180 captures sounds emitted from one or more sound sources in the local area (e.g., a room). Each acoustic sensor is configured to detect sound and convert the detected sound into an electronic format (analog or digital). The acoustic sensors 180 may be acoustic wave sensors, microphones, sound transducers, or similar sensors that are suitable for detecting sounds.

In some embodiments, one or more acoustic sensors 180 may be placed in an ear canal of each ear (e.g., acting as binaural microphones). In some embodiments, the acoustic sensors 180 may be placed on an exterior surface of the headset 100, placed on an interior surface of the headset 100, separate from the headset 100 (e.g., part of some other device), or some combination thereof. The number and/or locations of acoustic sensors 180 may be different from what is shown in FIG. 1A. For example, the number of acoustic detection locations may be increased to increase the amount of audio information collected and the sensitivity and/or accuracy of the information. The acoustic detection locations may be oriented such that the microphone is able to detect sounds in a wide range of directions surrounding the user wearing the headset 100.

The audio controller 150 processes information from the sensor array as detected by the sensor array. The audio controller 150 may comprise a processor and a computer-readable storage medium. The audio controller 150 may be configured to generate direction of arrival (DOA) estimates, generate acoustic transfer functions (e.g., array transfer functions and/or head-related transfer functions), track the location of sound sources, form beams in the direction of sound sources, classify sound sources, generate sound filters for the speakers 160, or some combination thereof.

In some embodiments described herein, the audio controller 150 may receive eye-tracking information from an eye-tracking system. The audio controller 150 may perform one or more actions based on the eye-tracking information from the eye-tracking system. In some embodiments, the audio controller 150 may use the eye-tracking information to selectively emphasize/de-emphasize acoustic content received from the acoustic sensors 180.

The position sensor 190 generates one or more measurement signals in response to motion of the headset 100. The position sensor 190 may be located on a portion of the frame 110 of the headset 100. The position sensor 190 may include an inertial measurement unit (IMU). Examples of position sensor 190 include: one or more accelerometers, one or more gyroscopes, one or more magnetometers, another suitable type of sensor that detects motion, a type of sensor used for error correction of the IMU, or some combination thereof. The position sensor 190 may be located external to the IMU, internal to the IMU, or some combination thereof.

In some embodiments, the headset 100 may provide for simultaneous localization and mapping (SLAM) for a position of the headset 100 and updating of a model of the local area. For example, the headset 100 may include a passive camera assembly (PCA) that generates color image data. The PCA may include one or more RGB cameras that capture images of some or all of the local area. In some embodiments, some or all of the imaging devices 130 of the DCA may also function as the PCA. The images captured by the PCA and the depth information determined by the DCA may be used to determine parameters of the local area, generate a model of the local area, update a model of the local area, or some combination thereof. Furthermore, the position sensor 190 tracks the position (e.g., location and pose) of the headset 100 within the room. Additional details regarding the components of the headset 100 are discussed below in connection with FIG. 7.

FIG. 1B is a perspective view of a headset 105 implemented as a HMD, in accordance with one or more embodiments. In some embodiments, the headset 105 is a client device. In embodiments that describe an AR system and/or a MR system, portions of a front side of the HMD are at least partially transparent in the visible band (˜380 nm to 750 nm), and portions of the HMD that are between the front side of the HMD and an eye of the user are at least partially transparent (e.g., a partially transparent electronic display). The HMD includes a front rigid body 115 and a band 175. The headset 105 includes many of the same components described above with reference to FIG. 1A but modified to integrate with the HMD form factor. For example, the HMD includes a display assembly, a DCA, an audio system, and a position sensor 190. FIG. 1B shows the illuminator 140, a plurality of the speakers 160, a plurality of the imaging devices 130, a plurality of acoustic sensors 180, a plurality of electrodes 185 of an electrode assembly, and the position sensor 190. The speakers 160 may be part of a transducer array (not shown) that also includes tissue transducers (e.g., a bone conduction transducer or a cartilage conduction transducer). The speakers 160 are shown located in various locations, such as coupled to the band 175 (as shown), coupled to front rigid body 115, or may be configured to be inserted within the ear canal of a user. The electrodes of the electrode assembly may be located at various portions of the HMD such that they are in direct contact with the skin of the user.

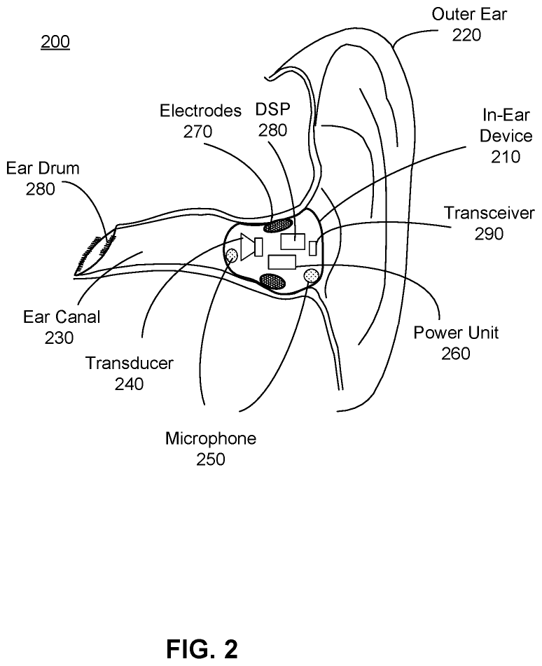

FIG. 2 is a profile view 200 of an in-ear device 210 to be used in conjunction with an eye-tracking system, in accordance with one or more embodiments. The in-ear device 210 may be component of wearable device assembly that includes a headset such as embodiments of headset 100 of FIG. 1A or FIG. 1B, in accordance with one or more embodiments. The profile view 200 depicts an outer ear 220 and an ear canal 230 for providing context. Although FIG. 2 illustrates an embodiment for a left ear, in other embodiments, it may also be for a right ear or both ears. In embodiments where there are individual in-ear devices for the left and the right ears, they may be connected (e.g., by a cable) or they may be individual separate devices (that may be in wireless communication with each other and/or some other device).

Embodiments of the in-ear device 210 include a transducer 240 that is part of a transducer array of an audio system, microphones 250, a power unit 260, a plurality of EOG electrodes 270, a digital signal processor (DSP) 280, and a transceiver 290. In alternative configurations, different and/or additional components may be included in the in-ear device 210, such as a receiver or a transceiver, and an in-ear device controller. Additionally, in some embodiments, the functionality described in conjunction with one or more of the components shown in FIG. 2 may be distributed among the components in a different manner than described in conjunction with FIG. 2.

In some embodiments, the in-ear device 210 is configured to be located entirely within the ear canal 230 of the user. The in-ear device 210 is placed within the ear canal 230 such that its placement may occlude a portion of the ear canal 230 either entirely, as depicted in FIG. 2, or it may occlude the portion partially. The in-ear device 210 is configured to be located in the ear canal 230 so that one side of the in-ear device, i.e., the external side, faces the outer ear 220, while the other end of the in-ear device 210, i.e., the internal side, faces the inner ear portion, i.e., towards the ear drum 280. Thus, the in-ear device 210 is located in the ear canal 260 so that the internal side of the in-ear device 210 is closer to the ear drum 280 than the external side of the in-ear device 210. In some embodiments, the in-ear device 210 may have a pre-shaped body that is based on deep scan ear canal geometry data derived from a population of users to ensure better fit for users.

The in-ear device 210 includes a transducer 240 that converts instructions received from an audio system to provide audio content to the user. The transducer 240 may be a high-bandwidth audio transducer.

The microphones 250 may include an internal microphone and an external microphone. The internal microphone detects airborne acoustic pressure waves in the ear canal. The internal microphone may be located near the internal side of the in-ear device 210 such that it faces the inner ear portion, towards the ear drum 280. In some embodiments, the airborne acoustic pressure waves detected by the internal microphone is converted into electrical signals and then provided to the audio system to be subsequently used for audio feedback and tuning when providing audio content to the user. The external microphone detects airborne acoustic pressure waves in the outer ear portion. The external microphone is located near the external side of the in-ear device 210 device such that it faces the outer ear 220 of the user. In some embodiments, the airborne acoustic pressure waves detected by the external microphone is converted into electrical signals and then provided to the audio system to be subsequently used for tuning purposes when providing audio content to the user and/or for hear-through purposes. In embodiments described herein, the microphone 250 uses micro-electro-mechanical system (MEMs) technology, and may be any of: a binaural microphone, a vibration sensor, a piezoelectric accelerometer, a capacitive accelerometer, or some combination thereof.

The power unit 260 provides power to the in-ear device 210 which is used to activate the transducer 240, the microphone 250, the DSP 270, and other components needing power. In some embodiments, the power unit 260 may include a battery. In some embodiments, the battery may be a rechargeable battery.

The EOG electrodes 270 monitor biopotential signals generated on the surface of the user's head during eye-movements of the user. While FIG. 2 illustrates two electrodes, in other embodiments, there may be more electrodes located within the in-ear device 210. In some embodiments, the electrodes 270 are spatially distributed on the outer surface of the in-ear device 210. In some embodiments, the electrodes are located in the in-ear device such that they touch a skin surface at the ear canal and a conchal bowl region of the user. The electrodes may be a plurality of silver chloride electrodes, a plurality of iridium oxide electrodes on a titanium substrate, or a plurality of gold-plated electrodes. In some embodiments, the plurality of electrodes may be soft, flat, stretchable, and foldable for ease of location and use on the outer surface of the in-ear device 210. Biopotentials corresponding to the eye's activities, (i.e., EOG) are collected using the embedded electrodes 270 and using analog front end (AFE) units.

The electrodes 270 measure biopotential signals generated within a head of the user in response to ocular events such as eye movements by the user. The measured biopotential signals captured by the AFE are provided to the DSP 280. The electrodes 270 may communicate with the DSP 280 using wireless communication or some communication circuitry (not shown) within the in-ear device 210 connecting the electrodes 270 to the DSP 280.

The DSP 280 may receive the monitored biopotential signals from the electrodes 270 for further signal processing. The monitored signals may be received from the electrodes wirelessly or through communication circuitry within the in-ear device 210 connecting the electrodes 270 to the DSP 280. In some embodiments, the DSP 280 may process the received signals from the electrodes, including filtering the signals. The DSP 280 may include the analog-to digital converter (ADC) and digital to analog (DAC) converters. The DSP 280 may include an amplifier to amplify the received biopotential signals from the electrodes. The DSP 280 may include filters such as a bandpass filter and or low-pass or high-pass filters and a notch filter to remove noise from the received signals. Power Line Interference (PLI) noises can be removed using notch filters at 60 Hz and also notch filters at its subharmonics (30 Hz, 20 Hz, 15 Hz, etc.) and harmonics (120, 180, 240 Hz) frequencies. Subsequently, the DSP 280 may provide the processed signals to the transceiver 290 for transmission to the eye tracking system in the headset. The signals may be provided by the DSP 280 to the transceiver 290 either using wireless communication or through communication circuitry (not shown) connecting the DSP 280 to the transceiver 290.

The transceiver 290 communicates the monitored and optionally processed signals received from the in-ear device 210 to the eye-tracking system located on the headset. In some embodiments, the transceiver unit 290 may include an antenna, a Bluetooth unit, and other transceiver components.

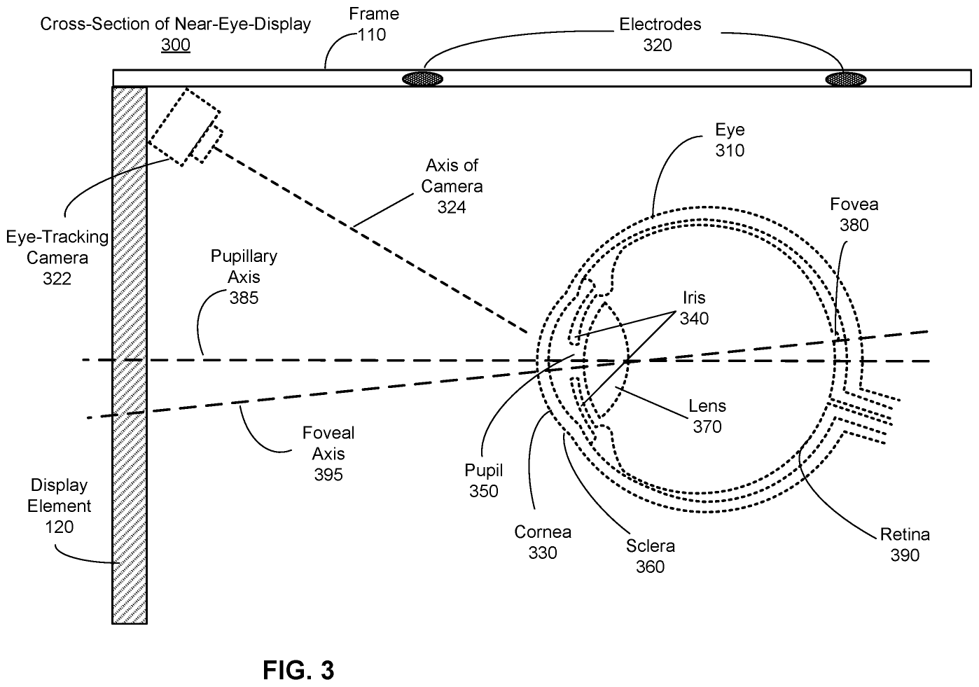

FIG. 3 is a cross section/side view 300 of a near-eye display, such as the headset 100 of FIG. 1A, relative to a user's eye 310, in accordance with one or more embodiments. Although FIG. 3 illustrates an embodiment for a one eye, in other embodiments, it may also and/or alternatively be for the other eye of the user. The cross-section of the near-eye display 300 includes a frame 110, a display element 120, electrodes 320, and an optionally included eye-tracker camera 322. The frame 110, the display element 120, and the electrodes 320 are embodiments of the frame 110, display element 120, and the electrodes 185 that are described with respect to FIG. 1A. In some embodiments, the eye-tracker camera 322 may be optionally included in the near-eye display 300 as an additional component of an eye-tracking system (not shown).

The eye 310 includes a cornea 330, an iris 340, a pupil 350, a sclera 360, a lens 370, a fovea 380, and a retina 390. The cornea 330 is the curved surface covering the iris 340 and the pupil 350 of the eye. The cornea 330 is essentially transparent in the visible band (˜380 nm to 750 nm) of the electromagnetic spectrum, and the near-infrared region (up to approximately 1,400 nanometers). The sclera 360 is the relatively opaque (usually visibly white) outer portion of the eye 310, which is often referred to as the “white of the eye.” The lens 370 is a transparent structure which serves to focus light at the retina 390 at the back of the eye 310. The iris 340 is a thin, colored, circular diaphragm concentric with the pupil 350. The iris 340 is the colored portion of the eye which contracts to alter the size of the pupil 350, a circular hole through which light enters the eye 310. The fovea 380 is an indent on the retina 390. The fovea 380 corresponds to the area of highest visual acuity for the user.

The eye's pupillary axis 385 and foveal axis 395 are depicted in FIG. 3. The pupillary axis 385 and foveal axis 395 change as the eye 310 moves. In FIG. 3, the eye 310 is depicted with a horizontal pupillary axis 385. Accordingly, the foveal axis 395 in FIG. 3 points about 6° below the horizontal plane. FIG. 3 also depicts the axis of the camera 324. FIG. 3 depicts an embodiment in which the eye-tracking camera 322 is not on either the pupillary axis 385 or the foveal axis 395. The camera 322 may be outside the visual field of the eye 310.

The movement of the eye 310 results in corresponding movements of corneal reflections at different gaze positions. These movements are captured by the eye-tracking camera 322. The captured movements are reported as eye movements by the eye-tracking camera 322 to an eye-tracking system (not shown). However, there are some disadvantages to using an eye-tracking camera such as camera 322. Some of the disadvantages include higher power requirements, occlusive effects such as during eye blinks, and low sampling frequencies. These disadvantages may be overcome with the use of the electrodes 320.

The EOG electrodes 320 are placed on the frame 110 such that they come into contact with the skin at the user's head. These electrodes 320 monitor the voltage potential difference (i.e., the biopotential signal) between the cornea 330 and the retina of the eye 310. As the eye 310 moves, the vector of the voltage potential difference between the cornea 330 and the retina 390 changes with respect to the EOG electrodes 320. As a consequence, the monitored signals at the electrodes 320 change, and may therefore be used to determine the eye movements. For example, during periods of open eyes, sharp deflections in the monitored signals at the electrodes 320 may be caused by eye blinks.

In some embodiments, the electrodes 320 may be located on the end pieces of the frame 110 so that they come in contact with the skin at the head of the user near the temple. In some embodiments, an electrode 320 may also be located on the frame where the frame bridges the nose of the user, where the electrode 320 may come in contact with the skin at the nose-bridge of the user. In some embodiments, the electrodes 320 may be placed on the frame 110 above and below the eye 310 such that they may come into contact with the skin on the forehead region above the eye 310 and a facial cheek region below the eye 310. Such electrode placement may facilitate the determination of vertical eye movements by the user. As the spacing between electrodes 320 increases, the measured signals may be less susceptible to noise related variations. It is therefore beneficial to have electrodes 320 distributed as spatially apart as possible in the frame 110 while still being able to obtain contact with the skin at the user's head. The monitored readings from the EOG electrodes 320 are reported to an eye-tracking system.

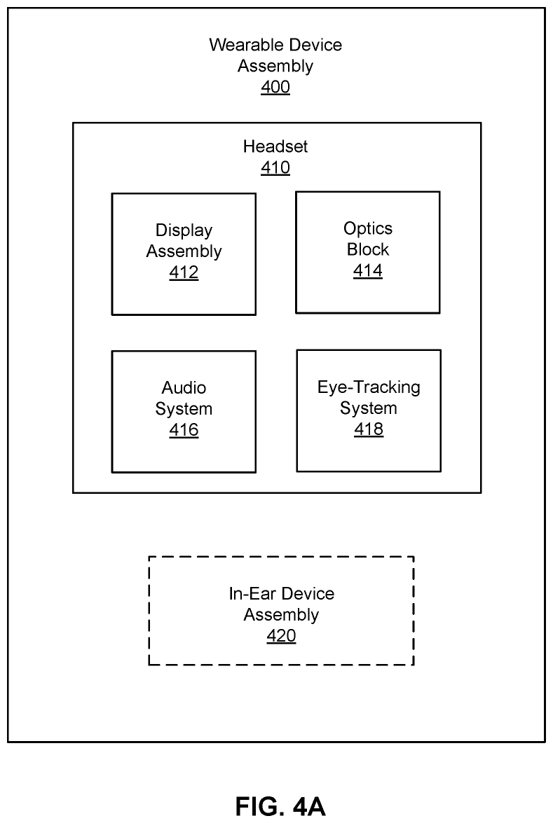

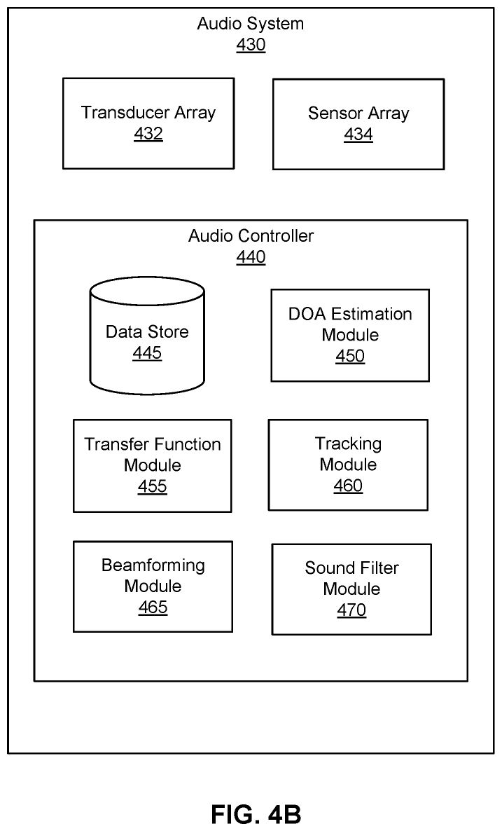

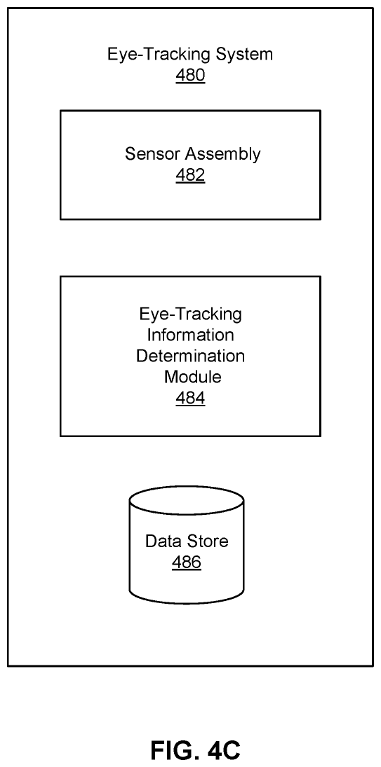

FIG. 4A is a block diagram of a wearable device assembly 400, in accordance with one or more embodiments. The wearable device assembly 400 includes a headset 410 and an in-ear device assembly 420. The in-ear device assembly 420 includes one in-ear device or two in-ear devices (i.e., one for each ear). The headset 100 depicted in FIG. 1A or the headset 105 depicted in FIG. 1B may be embodiments of the headset 410. The in-ear device 210 depicted in FIG. 2 may be an embodiment of the in-ear device 430. Some embodiments of the wearable device assembly 400 may include the in-ear device 430 while other embodiments of the wearable device assembly 400 may not include the in-ear device 430.

The headset 410 may include a display assembly 412, an optics block 414, an audio system 416 and an eye-tracking system 418. Some embodiments of the headset 410 may have different components than those described here. Similarly, in some cases, functions can be distributed among the components in a different manner than is described here.

The display assembly 412 displays content to the user in accordance with received instructions from a console (not shown). The display assembly 412 displays the content using one or more display elements. The display element 120 described in FIG. 1A and FIG. 3 may be embodiments of display elements in the display assembly 412. A display element may be an electronic display. In various embodiments, the display assembly 412 comprises a single display element or multiple display elements (e.g., a display for each eye of a user). Note in some embodiments, a display element may also include some or all of the functionality of the optics block 414.

In some embodiments, the display assembly 412 may receive eye-tracking information from eye-tracking system 418 about occurrence of an ocular event, for example ocular fixation. The display assembly 412 may use the received eye-tracking information to modify the displayed visual content to the user. For example, the eye-tracking system may determine, based on monitored biopotential signals and eye tracking camera information, that the eye-gaze of the user is fixed in a particular direction. Such information about ocular fixation in a particular direction may cause the display assembly 412 to modify the visual content presented to the user in a particular region of the displayed content. Other ocular events detected apart from ocular fixation may include ocular saccades, ocular blinks, ocular movement direction, and ocular movement speed. In some embodiments, information about ocular movement speed may be used by the display assembly 412 to modify the display based on predicted eye movement.

The optics block 414 may magnify image light received from the electronic display, corrects optical errors associated with the image light, and presents the corrected image light to one or both eye boxes (not shown) of the headset 410. In various embodiments, the optics block 414 includes one or more optical elements, or combinations of different optical elements. Magnification and focusing of the image light by the optics block 414 allows the electronic display to be physically smaller, weigh less, and consume less power than larger displays. Additionally, magnification may increase the field of view of the content presented by the electronic display.

In some embodiments, the optics block 414 may receive eye-tracking information from eye-tracking system 418 about occurrence of an ocular event, for example ocular fixation. The optics block 414 may use the received eye-tracking information to modify the displayed visual content to the user. For example, the eye-tracking system 418 may determine, based on monitored biopotential signals and eye tracking camera information, that the eye-gaze of the user is fixed in a particular direction. Such information about ocular fixation in a particular direction may cause the optics block 414 to modify the image presentation such that the image is presented at a particular image plane. The chosen image plane for presentation of the image is the image plane where the eye is determined to be currently focused. Other ocular events detected apart from ocular fixation may include ocular saccades, ocular blinks, ocular movement direction, and ocular movement speed. In some embodiments, information about ocular movement speed may be used by the optics block 414 to modify the display based on predicted eye movement.

The audio system 416 generates and presents audio content for the user. The audio system of FIG. 1A or FIG. 1B may be embodiments of the audio system 416. The audio system 416 may present audio content to the user through a transducer array (not shown) and/or the in-ear device assembly 420. In some embodiments of the audio system 416, the generated audio content is spatialized. Spatialized audio content is audio content that appears to originate from a particular direction and/or target region (e.g., an object in the local area and/or a virtual object). In some embodiments, the audio system 416 may combine information from different acoustic sensors to emphasize sound associated from a particular region of the local area while deemphasizing sound that is from outside of the region. In some embodiments, the audio system 416 receives eye-tracking information from the eye-tracking system 418 and uses this information to selectively emphasize and de-emphasize sound from various sources. For example, the eye-tracking system 418 may determine that the eye-gaze of the user is fixed in a particular direction. In some embodiments, the audio system 416 may selectively emphasize acoustic content associated with a particular region of a local area and to selectively deemphasize acoustic content that is from outside of the particular region based on the eye-gaze information.

The eye-tracking system 418 tracks eye movements of a user of the wearable device assembly 400. In some embodiments, the eye-tracking system 418 receives information about monitored biopotential signals from a plurality of EOG electrodes located on a headset (e.g., electrodes 185 in FIG. 1A, FIG. 1B, and FIG. 3). In some embodiments, the eye-tracking system 418 receives information about monitored biopotential signals from a plurality of EOG electrodes located within the optional in-ear device 420 (e.g., electrodes 270 in FIG. 2). In some embodiments, the eye-tracking system 418 may combine the information received from electrodes located on the headset (e.g., electrodes 185) and electrodes located in an included in-ear device (e.g., electrodes 270). In some embodiments, in addition to a plurality of electrodes such as described herein, the eye-tracking system 418 may include one or more eye-tracking cameras (e.g., eye-tracking camera 322). In these embodiments, the eye-tracking system 418 may combine the eye-tracking information determined from the monitored biopotential signals with the eye-tracking information received from the eye-tracking camera.

The eye-tracking system 418 may determine, based on the tracked eye movements, that the user's eye(s) exhibit occurrence of ocular events such as ocular saccade, ocular fixation, ocular blink, and ocular movement in a particular direction and/or at a particular speed. The eye-tracking system 418 may provide information about these determined ocular events to the display assembly 412 and/or the optics bock 414 as well as the audio system 416. In some embodiments, the eye-tracking system 418 may provide information about these determined ocular events to other components of the headset.

The wearable device assembly 400 may optionally include the in-ear device assembly 420 with one or more in-ear devices. The in-ear devices may be embodiments of the in-ear device 210 depicted in FIG. 2. An in-ear device includes a plurality of electrodes (e.g., electrodes 270) that are spatially distributed on an outer surface of the in-ear device and are contact with the surface of the ear canal and the surface of the conchal bowl region of the user's ear. The monitored biopotential signals received by the electrodes in the in-ear device assembly 420 may be sent to the eye-tracking system 418.

FIG. 4B is a block diagram of an audio system 430, in accordance with one or more embodiments. The audio system 416 depicted in FIG. 4A may be an embodiment of the audio system 480. The audio system 430 generates one or more acoustic transfer functions for a user. The audio system 430 may then use the one or more acoustic transfer functions to generate audio content for the user. In the embodiment of FIG. 4B, the audio system 430 includes a transducer array 432, a sensor array 434, and an audio controller 440. Some embodiments of the audio system 430 have different components than those described here. Similarly, in some cases, functions can be distributed among the components in a different manner than is described here.

The transducer array 432 is configured to present audio content. The transducer array 432 includes a plurality of transducers. A transducer is a device that provides audio content. A transducer may be, e.g., a speaker (e.g., the speaker 160), a tissue transducer (e.g., the tissue transducer 170), some other device that provides audio content, or some combination thereof. A tissue transducer may be configured to function as a bone conduction transducer or a cartilage conduction transducer. The transducer array 432 may present audio content via air conduction (e.g., via one or more speakers), via bone conduction (via one or more bone conduction transducer), via cartilage conduction audio system (via one or more cartilage conduction transducers), or some combination thereof. In some embodiments, the transducer array 432 may include one or more transducers to cover different parts of a frequency range. For example, a piezoelectric transducer may be used to cover a first part of a frequency range and a moving coil transducer may be used to cover a second part of a frequency range.

The bone conduction transducers generate acoustic pressure waves by vibrating bone/tissue in the user's head. A bone conduction transducer may be coupled to a portion of a headset, and may be configured to be behind the auricle coupled to a portion of the user's skull. The bone conduction transducer receives vibration instructions from the audio controller 330, and vibrates a portion of the user's skull based on the received instructions. The vibrations from the bone conduction transducer generate a tissue-borne acoustic pressure wave that propagates toward the user's cochlea, bypassing the eardrum.

The cartilage conduction transducers generate acoustic pressure waves by vibrating one or more portions of the auricular cartilage of the ears of the user. A cartilage conduction transducer may be coupled to a portion of a headset, and may be configured to be coupled to one or more portions of the auricular cartilage of the ear. For example, the cartilage conduction transducer may couple to the back of an auricle of the ear of the user. The cartilage conduction transducer may be located anywhere along the auricular cartilage around the outer ear (e.g., the pinna, the tragus, some other portion of the auricular cartilage, or some combination thereof). Vibrating the one or more portions of auricular cartilage may generate: airborne acoustic pressure waves outside the ear canal; tissue born acoustic pressure waves that cause some portions of the ear canal to vibrate thereby generating an airborne acoustic pressure wave within the ear canal; or some combination thereof. The generated airborne acoustic pressure waves propagate down the ear canal toward the ear drum.

In some embodiments, the audio content is spatialized. Spatialized audio content is audio content that appears to originate from a particular direction and/or target region (e.g., an object in the local area and/or a virtual object). For example, spatialized audio content can make it appear that sound is originating from a virtual singer across a room from a user of the audio system 430. The transducer array 432 may be coupled to a wearable device (e.g., the headset 410 in FIG. 4A). In alternate embodiments, the transducer array 432 may be a plurality of speakers that are separate from the wearable device (e.g., coupled to an external console).

The sensor array 320 detects sounds within a local area surrounding the sensor array 434. The sensor array 434 may include a plurality of acoustic sensors that each detect air pressure variations of a sound wave and convert the detected sounds into an electronic format (analog or digital). The plurality of acoustic sensors may be positioned on a headset (e.g., headset 410), on a user (e.g., in an ear canal of the user), on a neckband, or some combination thereof. An acoustic sensor may be, e.g., a microphone, a vibration sensor, an accelerometer, or any combination thereof. In some embodiments, the sensor array 434 is configured to monitor the audio content generated by the transducer array 310 using at least some of the plurality of acoustic sensors. Increasing the number of sensors may improve the accuracy of information (e.g., directionality) describing a sound field produced by the transducer array 432 and/or sound from the local area.

The audio controller 440 controls operation of the audio system 300. In the embodiment of FIG. 3, the audio controller 440 includes a data store 445, a DOA estimation module 450, a transfer function module 455, a tracking module 460, a beamforming module 465, and a sound filter module 470. The audio controller 440 may be located inside a headset, in some embodiments. Some embodiments of the audio controller 440 have different components than those described here. Similarly, functions can be distributed among the components in different manners than described here. For example, some functions of the controller may be performed external to the headset. The user may opt in to allow the audio controller 440 to transmit data captured by the headset to systems external to the headset, and the user may select privacy settings controlling access to any such data.

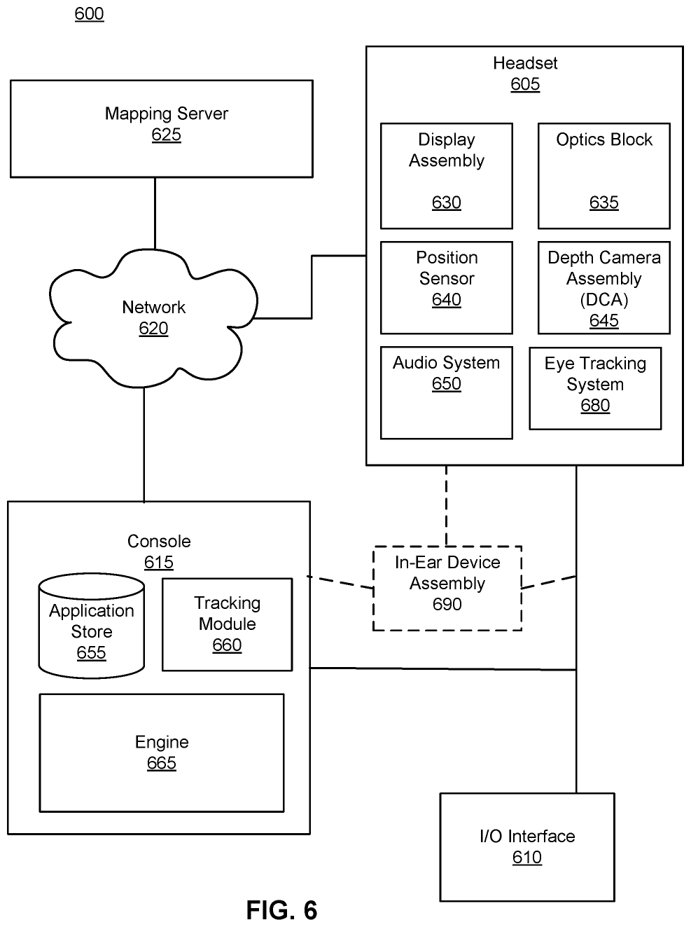

The data store 445 stores data for use by the audio system 430. Data in the data store 445 may include sounds recorded in the local area of the audio system 430, audio content, head-related transfer functions (HRTFs), transfer functions for one or more sensors, array transfer functions (ATFs) for one or more of the acoustic sensors, sound source locations, virtual model of local area, direction of arrival estimates, sound filters, and other data relevant for use by the audio system 430, or any combination thereof. Data in the data store 445 may also include data that is received from a server (e.g., the mapping server 625 in FIG. 6) for use by the audio system. In some embodiments, the data store 445 may store acoustic parameters that describe acoustic properties of the local area. The stored acoustic parameters may include, e.g., a reverberation time, a reverberation level, a room impulse response, etc.

The DOA estimation module 450 is configured to localize sound sources in the local area based in part on information from the sensor array 434. Localization is a process of determining where sound sources are located relative to the user of the audio system 430. The DOA estimation module 450 performs a DOA analysis to localize one or more sound sources within the local area. The DOA analysis may include analyzing the intensity, spectra, and/or arrival time of each sound at the sensor array 434 to determine the direction from which the sounds originated. In some cases, the DOA analysis may include any suitable algorithm for analyzing a surrounding acoustic environment in which the audio system 430 is located.

For example, the DOA analysis may be designed to receive input signals from the sensor array 434 and apply digital signal processing algorithms to the input signals to estimate a direction of arrival. These algorithms may include, for example, delay and sum algorithms where the input signal is sampled, and the resulting weighted and delayed versions of the sampled signal are averaged together to determine a DOA. A least mean squared (LMS) algorithm may also be implemented to create an adaptive filter. This adaptive filter may then be used to identify differences in signal intensity, for example, or differences in time of arrival. These differences may then be used to estimate the DOA. In another embodiment, the DOA may be determined by converting the input signals into the frequency domain and selecting specific bins within the time-frequency (TF) domain to process. Each selected TF bin may be processed to determine whether that bin includes a portion of the audio spectrum with a direct path audio signal. Those bins having a portion of the direct-path signal may then be analyzed to identify the angle at which the sensor array 434 received the direct-path audio signal. The determined angle may then be used to identify the DOA for the received input signal. Other algorithms not listed above may also be used alone or in combination with the above algorithms to determine DOA. In some embodiments, the DOA estimation module 450 may also determine the DOA with respect to an absolute position of the audio system 430 within the local area. The position of the sensor array 434 may be received from an external system (e.g., some other component of a headset, an artificial reality console, an audio server, a position sensor (e.g., the position sensor 190), etc.). The external system may create a virtual model of the local area, in which the local area and the position of the audio system 300 are mapped. The received position information may include a location and/or an orientation of some or all of the audio system 300 (e.g., of the sensor array 434). The DOA estimation module 450 may update the estimated DOA based on the received position information.

The transfer function module 455 is configured to generate one or more acoustic transfer functions. Generally, a transfer function is a mathematical function giving a corresponding output value for each possible input value. Based on parameters of the detected sounds, the transfer function module 455 generates one or more acoustic transfer functions associated with the audio system. The acoustic transfer functions may be array transfer functions (ATFs), head-related transfer functions (HRTFs), other types of acoustic transfer functions, or some combination thereof. An ATF characterizes how the microphone receives a sound from a point in space.

An ATF includes a number of transfer functions that characterize a relationship between the sound source and the corresponding sound received by the acoustic sensors in the sensor array 434. Accordingly, for a sound source there is a corresponding transfer function for each of the acoustic sensors in the sensor array 434. And collectively the set of transfer functions is referred to as an ATF. Accordingly, for each sound source there is a corresponding ATF. Note that the sound source may be, e.g., someone or something generating sound in the local area, the user, or one or more transducers of the transducer array 432. The ATF for a particular sound source location relative to the sensor array 434 may differ from user to user due to a person's anatomy (e.g., ear shape, shoulders, etc.) that affects the sound as it travels to the person's ears. Accordingly, the ATFs of the sensor array 434 are personalized for each user of the audio system 430.

In some embodiments, the transfer function module 455 determines one or more HRTFs for a user of the audio system 430. The HRTF characterizes how an ear receives a sound from a point in space. The HRTF for a particular source location relative to a person is unique to each ear of the person (and is unique to the person) due to the person's anatomy (e.g., ear shape, shoulders, etc.) that affects the sound as it travels to the person's ears. In some embodiments, the transfer function module 455 may determine HRTFs for the user using a calibration process. In some embodiments, the transfer function module 455 may provide information about the user to a remote system. The user may adjust privacy settings to allow or prevent the transfer function module 455 from providing the information about the user to any remote systems. The remote system determines a set of HRTFs that are customized to the user using, e.g., machine learning, and provides the customized set of HRTFs to the audio system 430.

The tracking module 460 is configured to track locations of one or more sound sources. The tracking module 460 may compare current DOA estimates and compare them with a stored history of previous DOA estimates. In some embodiments, the audio system 430 may recalculate DOA estimates on a periodic schedule, such as once per second, or once per millisecond. The tracking module may compare the current DOA estimates with previous DOA estimates, and in response to a change in a DOA estimate for a sound source, the tracking module 460 may determine that the sound source moved. In some embodiments, the tracking module 460 may detect a change in location based on visual information received from the headset or some other external source. The tracking module 460 may track the movement of one or more sound sources over time. The tracking module 460 may store values for a number of sound sources and a location of each sound source at each point in time. In response to a change in a value of the number or locations of the sound sources, the tracking module 460 may determine that a sound source moved. The tracking module 460 may calculate an estimate of the localization variance. The localization variance may be used as a confidence level for each determination of a change in movement.

The beamforming module 465 is configured to process one or more ATFs to selectively emphasize sounds from sound sources within a certain area while de-emphasizing sounds from other areas. In analyzing sounds detected by the sensor array 434, the beamforming module 465 may combine information from different acoustic sensors to emphasize sound associated from a particular region of the local area while deemphasizing sound that is from outside of the region. The beamforming module 465 may isolate an audio signal associated with sound from a particular sound source from other sound sources in the local area based on, e.g., different DOA estimates from the DOA estimation module 450 and the tracking module 460. The beamforming module 465 may thus selectively analyze discrete sound sources in the local area. In some embodiments, the beamforming module 465 may enhance a signal from a sound source. For example, the beamforming module 465 may apply sound filters which eliminate signals above, below, or between certain frequencies. Signal enhancement acts to enhance sounds associated with a given identified sound source relative to other sounds detected by the sensor array 434.

In some embodiments, the beamforming module 465 may receive eye-tracking information from the eye-tracking system (e.g., eye-tracking system 418 in FIG. 4A) and use this information to selectively emphasize and de-emphasize sound from various sources. For example, the eye-tracking system may determine that the eye-gaze of the user is fixed in a particular direction. In some embodiments, the beamforming module 465 may selectively emphasize acoustic content associated with a particular region of a local area and to selectively deemphasize acoustic content that is from outside of the particular region based on the eye-gaze information. The beamforming module 465 may combine information from the one or more acoustic sensors in the sensor array 434 to perform the selective emphasizing and deemphasizing of acoustic content.

The sound filter module 470 determines sound filters for the transducer array 432. In some embodiments, the sound filters cause the audio content to be spatialized, such that the audio content appears to originate from a target region. The sound filter module 470 may use HRTFs and/or acoustic parameters to generate the sound filters. The acoustic parameters describe acoustic properties of the local area. The acoustic parameters may include, e.g., a reverberation time, a reverberation level, a room impulse response, etc. In some embodiments, the sound filter module 470 calculates one or more of the acoustic parameters. In some embodiments, the sound filter module 470 may generate spatial signal enhancement filters based on the calculated acoustic parameters to provide to the transducer array 432.

FIG. 4C is a block diagram of an eye-tracking system 480, in accordance with one or more embodiments. The eye-tracking system 480 is an embodiment of the eye-tracking system 418 depicted in FIG. 4A. The eye-tracking system 480 may include a sensor assembly 482, an eye-tracking information determination module 484, and a data store 486. Some embodiments of the eye-tracking system 480 may have different components than those described here. Similarly, in some cases, functions can be distributed among the components in a different manner than is described here.

The sensor assembly 482 includes a plurality of sensors that detect information related to eye movements of the user of a wearable device assembly 400 such as depicted in FIG. 4A. The plurality of sensors in the sensor assembly 482 may include a plurality of EOG electrodes that monitor biopotential signals generated on a user's head. The sensor assembly 482 may also include one or more eye-tracking cameras that detect and track corneal reflections at different gaze positions in the user's eye.

The plurality of electrodes in the sensor assembly 482 monitor biopotential signals that are generated within a head of the user in response to the occurrence of ocular events such as eye movements, saccades, eye blinks, etc. As described with respect to the user eye depicted in FIG. 3, these electrodes monitor the voltage potential difference (i.e., the biopotential signal) between the cornea and the retina of the eye. As the eye moves, the vector of the voltage potential difference between the cornea and the retina changes with respect to the electrodes. As a consequence, the monitored signals at the electrodes change, and may therefore be used to further determine the eye movements. The measured signals are sent by the sensor assembly 482 to the eye-tracking controller module 484 for determining eye-tracking information.

The electrodes are coupled to and/or attached to different portions of the wearable device assembly and are in direct contact with skin of the user. In some embodiments, the plurality of electrodes in the sensor assembly 482 may be located on a headset alone, on one or more in-ear devices alone, or on both a headset and one or more in-ear devices.

In some embodiments, the electrodes may be located on a headset. The electrodes 185 in FIG. 1A, FIG. 1B, and FIG. 3 are embodiments of the electrodes in sensor assembly 482. As illustrated in FIG. 1A, FIG. 1B, and FIG. 3, the plurality of electrodes may be spatially distributed on the frame, at the nose bridge, as well as the end pieces of the frame. In some embodiments, the plurality of electrodes includes a ground electrode that is mounted on a front part of the frame of the headset. In some embodiments, the electrodes may be spatially distributed on other portions of a headset including, e.g., portions of a frame of a headset, the temples of the frame, a bridge of the frame, a band of the headset, portions in contact with the nose, portions in contact with the forehead, or some other portion of the headset or some combination thereof. As the spacing between electrodes increases, the measured signals may be less susceptible to noise related variations. It is therefore beneficial to have electrodes distributed as spatially apart as possible on the frame while still being able to obtain contact with the skin at the user's head. In some embodiments, the plurality of electrodes in the sensor assembly 482 include electrodes that are mounted on the headset to be in contact with a forehead region above an eye of the user, and electrodes that are mounted on the headset to be in contact with a facial cheek region below an eye of the user. Such a configuration of electrodes on the headset facilitates the determination of up-down eye movements (i.e., eye movements that are orthogonal to side-to-side eye movements by the user).

In some embodiments, the plurality of electrodes in the sensor assembly 482 may be located as portions of in-ear devices, portions of hearing aids, portions of hearables, or some combination thereof. The electrodes 270 in FIG. 2 are an embodiment of the electrodes in the sensor assembly 482 that are located in an in-ear device. In these embodiments, the plurality of electrodes includes electrodes that are spatially distributed on an outer surface of the in-ear device. Furthermore, the plurality of electrodes includes electrodes that are located on the outer surface of the in-ear device and that touch an ear canal region and a conchal bowl region of the user.

Embodiments of the plurality of electrodes in the sensor assembly 482 are EOG electrodes that include a plurality of silver chloride electrodes, a plurality of iridium oxide electrodes on a titanium substrate, or a plurality of gold-plated electrodes. In some embodiments, the plurality of electrodes may be soft, flat, and foldable for ease of location and use on the headset or on an in-ear device.

In some embodiments, the sensor assembly 482 may also include one or more eye-tracking cameras. The eye-tracking camera 320 in FIG. 3 may be an embodiment of the eye tracking cameras in the sensor assembly 482. The one or more eye-tracking cameras track the eye movements based on detecting corneal reflections at different gaze positions. In some embodiments, the eye-tracking cameras may be infrared cameras (i.e., a camera designed to capture images in the infrared frequency). In some embodiments, the eye-tracking cameras may be a near-infrared camera with digital image sensors. The eye-tracking cameras may include a CCD or CMOS digital image sensor and an optical element. The optical element may be one or more lenses, a high-pass, low-pass, or band-pass filter, a polarizer, an aperture stop, a diaphragm, some other optical element suitable for processing IR light, or some combination thereof. The optical element outputs light which is captured and converted into a digital signal by the CCD or CMOS digital sensor.

In some embodiments, the sensor assembly may also include a signal processing unit to process the monitored biopotential signals received from the plurality of electrodes prior to providing them to the eye-tracking information determination module 484. The signal processing unit may process the received signals from the electrodes, the processing including filtering the signals using a bandpass filter and a notch filter to remove noise from the received signals. The filters may be tuned to perform filtering such that the signal to noise ratio in the signals is above a prespecified target threshold. In some embodiments, the unit may amplify the received biopotential signals from the electrodes.

The eye-tracking information determination module 484 determines eye-tracking information for the user using a machine learning model based on the monitored biopotential signals. The determined eye-tracking information may include the occurrence and identification of ocular events such as ocular fixation, ocular saccades, ocular blinks, ocular movement in a particular direction and/or a particular speed.

The eye-tracking information determination module 484 receives the monitored biopotential information from the sensor assembly 482. The biopotential signals monitored by the plurality of electrodes are obtained at a higher sampling frequency than a sampling frequency used to track eye movements by the eye-tracking camera. Thus, in embodiments where both the electrodes and the eye-tracking camera are present in the sensor assembly 482, for a same period of monitored time, more eye-tracking related information is received from the electrodes than from the eye-tracking cameras. Hence, information that is obtained from the plurality of electrodes may be used to compensate for missing information in the received eye-tracking information from the eye-tracking cameras, and thereby generate improved eye-tracking information. Thus, the eye-tracking information from the electrodes provide more eye-tracking information (i.e., at a finer resolution) than the eye-tracking information from the eye-tracking cameras within any given period of time for use in determining eye-movement information during that period of time. Thus, in some embodiments where both the electrodes and the eye-tracking camera are present in the sensor assembly 482, concurrent to monitoring the biopotential signals from the plurality of electrodes, the eye-tracking information determination module 484 is also receiving information regarding eye movements of the user from one or more eye-tracking sensors mounted on the device. The eye-tracking information determination module 484 combines the information regarding eye movements of the user from the eye-tracking cameras with the determined eye-tracking information based on the monitored biopotential signals to generate improved eye-tracking information. Similarly, in some situations the information obtained from the eye-tracking cameras is of low quality—such as with eyelid occlusions, dark environments, low power availability, etc. In such situations, the eye-tracking information determination module 484 combines the information regarding eye movements of the user from the eye-tracking cameras with the determined eye-tracking information based on the monitored biopotential signals to generate improved eye-tracking information.

The eye-tracking information determination module 484 receives the monitored biopotential information from the sensor assembly 482. However, the EOG electrodes used in the sensor assembly 482 may exhibit signal drift due to a potential that is built up between an electrode and a region of the skin that the electrode is in contact with. This signal drift that may be present in the received biopotential signal information from the sensor assembly 482 may be corrected with the use of information from the eye-tracking cameras. Thus, in some embodiments where both the electrodes and the eye-tracking camera are present in the sensor assembly 482, concurrent to monitoring the biopotential signals from the plurality of electrodes, the eye-tracking information determination module 484 receives information regarding eye movements of the user from the eye tracking camera and compares the information regarding eye movements of the user with the determined eye-tracking information based on the monitored biopotential signals. Based on the comparison, the eye-tracking information determination module 484 determines that the monitored biopotential signals from the plurality of electrodes exhibit signal drift and corrects the determined signal drift in the monitored biopotential signals using one or more signal filters (e.g., using high-pass filters).

The eye-tracking information determination module 484 may determine eye-tracking information for the user using a trained machine learning model based on the monitored biopotential signals. In some embodiments, the machine learning model may be obtained by the module 484 from the data store 486. In some embodiments, the eye-tracking information determination module 484 may use the trained machine learning model to determine the occurrence of ocular events such as ocular saccades, ocular blinks, ocular fixation, ocular movements in particular direction and/or at a particular speed, etc., based on a stored mapping between the ocular events and the monitored biopotential signals. In some embodiments, the model mapping may also provide the eye-tracking information determination module 484 with a prediction metric such as an associated probability of occurrence of the ocular event. The associated probability may be based on an estimate of the signal to noise ratio of the monitored biopotential signal.

In some embodiments, the eye-tracking information determination module 484 may periodically request a model from a mapping server. In response to the request, the module 484 may receive a possibly updated model from the mapping server through a network and store the model at the data store 486. In some embodiments, the module 484 may periodically receive an updated model from the mapping server through the network without having to request the mapping server.

The determined eye-tracking information from the eye-tracking information determination module 484 may be provided to various components of the headset where they may be used to perform actions by these components. Examples of such components of the headset include a display assembly (e.g., display assembly 412 in FIG. 4A), an optics block (e.g., the optics block 414 in FIG. 4A), and an audio system (e.g., audio system 416 in FIG. 4A). The actions performed by the display assembly and the optics block may include adjusting a display of visual content presented to the user based on the information associated with the determined one or more ocular events. The actions performed by the audio system may include using the eye-tracking information to selectively emphasize/de-emphasize acoustic content received at acoustic sensors. For example, the user may be located in a crowded environment where there are different competing talkers and other acoustic content. The user may wish to hear and attend to acoustic content coming from a specific direction/location that they are seeing. The acoustic content that is not coming from that particular location/direction needs to be attenuated. The audio system uses the determined eye-tracking information (i.e., where the user is directing their attention), and using this information to steer the output of the audio system to enhance the acoustic content pick-up in the specific direction of attention.

The data store 486 stores data for use by the eye-tracking system 480. In some embodiments, the data in the data store 486 includes model information that is generated and provided by a mapping server (e.g., mapping server 625 in FIG. 6). In some embodiments, the model information may be associated with a trained machine learning model that is received from the server. The model information provides a mapping between the monitored biopotential signals generated by the plurality of electrodes in the sensor assembly 482 and eye-tracking information parameter values. In some embodiments, the model information may be in the form of one or more look-up tables that map biopotential signals to particular ocular events such as ocular saccades, ocular blinks, ocular movement in particular direction and/or speed, ocular fixation, etc. In some embodiments, the look-up tables may be generated from the trained machine learning model. In some embodiments, the data store 488 may store prespecified threshold values such as target signal to noise ratios for the measured biopotential signals, etc.

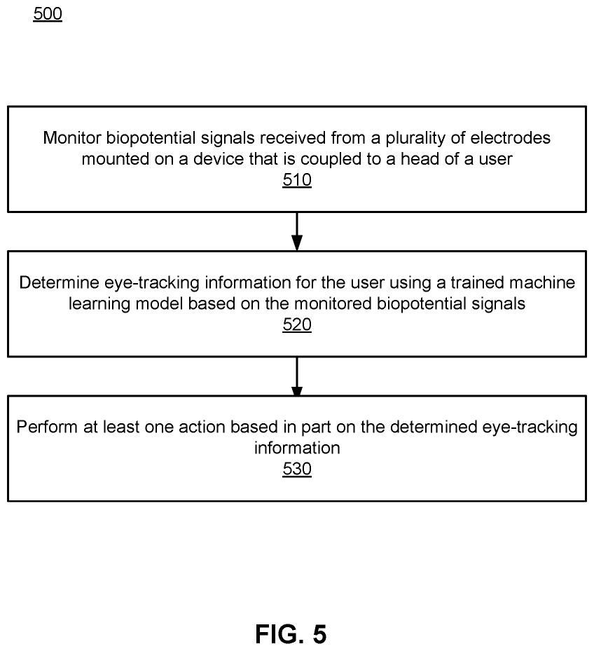

FIG. 5 is a flowchart for using eye-tracking information, in accordance with one or more embodiments. The process shown in FIG. 5 may be performed by wearable device assembly. Other entities may perform some or all of the steps in FIG. 5 in other embodiments. Embodiments may include different and/or additional steps or perform the steps in different orders.

The wearable device assembly monitors 510 (e.g., via an eye tracking system) biopotential signals that are received from a plurality of electrodes mounted on a device that is coupled to a head of a user. The biopotential signals are monitored from electrodes that may be spatially distributed on an outer surface of an in-ear device (e.g., such that they are in contact with an ear canal region or a conchal bowl region of the user), spatially distributed on a headset (e.g., such as on the frame of the headset where they are in contact with the head of the user in the temple region, the nose bridge region and/or regions above and below an eye of the user), or some combination thereof.

The wearable device assembly determines 520 eye-tracking information for the user using a trained machine learning model based on the monitored biopotential signals. The model may be a mapping of various biopotential signal values to corresponding one or more eye tracking information parameter values. The mapping may be stored as one or more look-up tables. The wearable device assembly may determine the eye tracking information parameter values for the monitored biopotential signals by retrieving the eye tracking information parameter values from the stored look-up tables. In some embodiments, the model may be a machine learning model that is trained at a remote location. The trained machine learning model may be stored at a mapping server, and the stored one or more look-up tables are generated from the trained machine learning model and stored at the mapping server from which the wearable device assembly may retrieve them.

The wearable device assembly performs 530 at least one action based in part on the determined eye-tracking information. In some embodiments, the actions performed 530 by the wearable device assembly may include adjusting a display of visual content presented to the user based on the information associated with the determined one or more ocular events, using the eye-tracking information to selectively emphasize/de-emphasize acoustic content received at acoustic sensors, or some combination thereof.

FIG. 6 is a system 600 that includes a headset 605, in accordance with one or more embodiments. In some embodiments, the headset 605 may be the headset 100 of FIG. 1A or the headset 105 of FIG. 1B. In some embodiments, the headset 605 may be a client device. The system 600 may operate in an artificial reality environment (e.g., a virtual reality environment, an augmented reality environment, a mixed reality environment, or some combination thereof). The system 600 shown by FIG. 6 includes the headset 605, an optional in-ear device assembly 690 that may include one or more in-ear devices, an input/output (I/O) interface 610 that is coupled to a console 615, the network 620, and the mapping server 625. While FIG. 6 shows an example system 600 including one headset 605 and one I/O interface 610, in other embodiments any number of these components may be included in the system 600. For example, there may be multiple headsets each having an associated I/O interface 610, with each headset and I/O interface 610 communicating with the console 615. In alternative configurations, different and/or additional components may be included in the system 600. Additionally, functionality described in conjunction with one or more of the components shown in FIG. 6 may be distributed among the components in a different manner than described in conjunction with FIG. 6 in some embodiments. For example, some or all of the functionality of the console 615 may be provided by the headset 605.

The headset 605 includes the display assembly 630, an optics block 635, one or more position sensors 640, a DCA 645, an audio system 650, and an eye-tracking system 680. Some embodiments of headset 605 have different components than those described in conjunction with FIG. 6. Additionally, the functionality provided by various components described in conjunction with FIG. 6 may be differently distributed among the components of the headset 605 in other embodiments or be captured in separate assemblies remote from the headset 605.

The display assembly 630 displays content to the user in accordance with data received from the console 615. The display assembly 630 displays the content using one or more display elements (e.g., the display elements 120). A display element may be, e.g., an electronic display. In various embodiments, the display assembly 630 comprises a single display element or multiple display elements (e.g., a display for each eye of a user). Examples of an electronic display include: a liquid crystal display (LCD), an organic light emitting diode (OLED) display, an active-matrix organic light-emitting diode display (AMOLED), a waveguide display, some other display, or some combination thereof. Note in some embodiments, the display element 120 may also include some or all of the functionality of the optics block 635.

The optics block 635 may magnify image light received from the electronic display, corrects optical errors associated with the image light, and presents the corrected image light to one or both eyeboxes of the headset 605. In various embodiments, the optics block 635 includes one or more optical elements. Example optical elements included in the optics block 635 include: an aperture, a Fresnel lens, a convex lens, a concave lens, a filter, a reflecting surface, or any other suitable optical element that affects image light. Moreover, the optics block 635 may include combinations of different optical elements. In some embodiments, one or more of the optical elements in the optics block 635 may have one or more coatings, such as partially reflective or anti-reflective coatings.

Magnification and focusing of the image light by the optics block 635 allows the electronic display to be physically smaller, weigh less, and consume less power than larger displays. Additionally, magnification may increase the field of view of the content presented by the electronic display. For example, the field of view of the displayed content is such that the displayed content is presented using almost all (e.g., approximately 110 degrees diagonal), and in some cases, all of the user's field of view. Additionally, in some embodiments, the amount of magnification may be adjusted by adding or removing optical elements.

In some embodiments, the optics block 635 may be designed to correct one or more types of optical error. Examples of optical error include barrel or pincushion distortion, longitudinal chromatic aberrations, or transverse chromatic aberrations. Other types of optical errors may further include spherical aberrations, chromatic aberrations, or errors due to the lens field curvature, astigmatisms, or any other type of optical error. In some embodiments, content provided to the electronic display for display is pre-distorted, and the optics block 635 corrects the distortion when it receives image light from the electronic display generated based on the content.

The position sensor 640 is an electronic device that generates data indicating a position of the headset 605. The position sensor 640 generates one or more measurement signals in response to motion of the headset 605. The position sensor 190 is an embodiment of the position sensor 640. Examples of a position sensor 640 include: one or more IMUS, one or more accelerometers, one or more gyroscopes, one or more magnetometers, another suitable type of sensor that detects motion, or some combination thereof. The position sensor 640 may include multiple accelerometers to measure translational motion (forward/back, up/down, left/right) and multiple gyroscopes to measure rotational motion (e.g., pitch, yaw, roll). In some embodiments, an IMU rapidly samples the measurement signals and calculates the estimated position of the headset 605 from the sampled data. For example, the IMU integrates the measurement signals received from the accelerometers over time to estimate a velocity vector and integrates the velocity vector over time to determine an estimated position of a reference point on the headset 605. The reference point is a point that may be used to describe the position of the headset 605. While the reference point may generally be defined as a point in space, however, in practice the reference point is defined as a point within the headset 605.

The DCA 645 generates depth information for a portion of the local area. The DCA includes one or more imaging devices and a DCA controller. The DCA 645 may also include an illuminator. Operation and structure of the DCA 645 is described above with regard to FIG. 1A.

The audio system 650 provides audio content to a user of the headset 605. The audio system 650 is substantially the same as the audio system 430 described with respect to FIG. 4B. The audio system 650 may include a sensor array with one or more acoustic sensors, a transducer array including one or more transducers, and an audio controller. In some embodiments described herein, the audio system 650 may receive eye-tracking information from the eye-tracking system 680. The audio system 650 may perform one or more actions based on the eye-tracking information from the eye-tracking system 680. In some embodiments, the audio system 650 may use the eye-tracking information to selectively emphasize/de-emphasize acoustic content.

The eye-tracking system 680 tracks eye movements of a user of the headset 605. The eye-tracking system 680 may include an electrode assembly with a plurality of EOG electrodes, one or more eye-tracking cameras, or some combination thereof. In some embodiments, the eye-tracking system 680 receives information about monitored biopotential signals from the plurality of EOG electrodes located on a headset, from an in-ear device assembly 690, or from some combination thereof. In some embodiments, the eye-tracking system 680 obtains additional information from one or more eye-tracking sensors that may also be part of the headset (e.g., the eye-tracking camera depicted in FIG. 3). The eye-tracking system 680 determines eye tracking information using a trained machine learning model based on the monitored eye-tracking information. The eye-tracking system 680 performs actions based on the determined eye-tracking information in conjunction with the display assembly 630, the optics block 635, and the audio system 650.

The in-ear device assembly 690 may have one or more in-ear devices that are configured to be located entirely within the ear canal of the user of the headset. An in-ear device includes a transducer that converts audio instructions received from an audio system into acoustic pressure vibrations in the ear canal, thereby providing audio content to the user. The in-ear device may be optionally worn by the user and is substantially similar to the in-ear device 210 described in FIG. 2. The in-ear device may include EOG electrodes that are in contact with ear canal and conchal regions of the user when worn. These electrodes measure biopotential signals generated within the head of the user in response to ocular events such as eye movements by the user. The in-ear device may transmit the monitored signals to the eye-tracking system 680.

The I/O interface 610 is a device that allows a user to send action requests and receive responses from the console 615. An action request is a request to perform a particular action. For example, an action request may be an instruction to start or end capture of image or video data, or an instruction to perform a particular action within an application. The I/O interface 610 may include one or more input devices. Example input devices include: a keyboard, a mouse, a game controller, or any other suitable device for receiving action requests and communicating the action requests to the console 615. An action request received by the I/O interface 610 is communicated to the console 615, which performs an action corresponding to the action request. In some embodiments, the I/O interface 610 includes an IMU that captures calibration data indicating an estimated position of the I/O interface 610 relative to an initial position of the I/O interface 610. In some embodiments, the I/O interface 610 may provide haptic feedback to the user in accordance with instructions received from the console 615. For example, haptic feedback is provided when an action request is received, or the console 615 communicates instructions to the I/O interface 610 causing the I/O interface 610 to generate haptic feedback when the console 615 performs an action.

The console 615 provides content to the headset 605 for processing in accordance with information received from one or more of: the DCA 645, the headset 605, and the I/O interface 610. In the example shown in FIG. 6, the console 615 includes an application store 655, a tracking module 660, and an engine 665. Some embodiments of the console 615 have different modules or components than those described in conjunction with FIG. 6. Similarly, the functions further described below may be distributed among components of the console 615 in a different manner than described in conjunction with FIG. 6. In some embodiments, the functionality discussed herein with respect to the console 615 may be implemented in the headset 605, or a remote system.

The application store 655 stores one or more applications for execution by the console 615. An application is a group of instructions, that when executed by a processor, generates content for presentation to the user. Content generated by an application may be in response to inputs received from the user via movement of the headset 605 or the I/O interface 610. Examples of applications include: gaming applications, conferencing applications, video playback applications, or other suitable applications.

The tracking module 660 tracks movements of the headset 605 or of the I/O interface 610 using information from the DCA 645, the one or more position sensors 640, or some combination thereof. For example, the tracking module 660 determines a position of a reference point of the headset 605 in a mapping of a local area based on information from the headset 605. The tracking module 660 may also determine positions of an object or virtual object. Additionally, in some embodiments, the tracking module 660 may use portions of data indicating a position of the headset 605 from the position sensor 640 as well as representations of the local area from the DCA 645 to predict a future location of the headset 605. The tracking module 660 provides the estimated or predicted future position of the headset 605 or the I/O interface 610 to the engine 665.

The engine 665 executes applications and receives position information, acceleration information, velocity information, predicted future positions, or some combination thereof, of the headset 605 from the tracking module 660. Based on the received information, the engine 665 determines content to provide to the headset 605 for presentation to the user. For example, if the received information indicates that the user has looked to the left, the engine 665 generates content for the headset 605 that mirrors the user's movement in a virtual local area or in a local area augmenting the local area with additional content. Additionally, the engine 665 performs an action within an application executing on the console 615 in response to an action request received from the I/O interface 610 and provides feedback to the user that the action was performed. The provided feedback may be visual or audible feedback via the headset 605 or haptic feedback via the I/O interface 610.

The network 620 couples the headset 605 and/or the console 615 to the mapping server 625. The network 620 may include any combination of local area and/or wide area networks using both wireless and/or wired communication systems. For example, the network 620 may include the Internet, as well as mobile telephone networks. In one embodiment, the network 620 uses standard communications technologies and/or protocols. Hence, the network 620 may include links using technologies such as Ethernet, 802.11, worldwide interoperability for microwave access (WiMAX), 2G/3G/4G mobile communications protocols, digital subscriber line (DSL), asynchronous transfer mode (ATM), InfiniBand, PCI Express Advanced Switching, etc. Similarly, the networking protocols used on the network 620 can include multiprotocol label switching (MPLS), the transmission control protocol/Internet protocol (TCP/IP), the User Datagram Protocol (UDP), the hypertext transport protocol (HTTP), the simple mail transfer protocol (SMTP), the file transfer protocol (FTP), etc. The data exchanged over the network 620 can be represented using technologies and/or formats including image data in binary form (e.g. Portable Network Graphics (PNG)), hypertext markup language (HTML), extensible markup language (XML), etc. In addition, all or some of links can be encrypted using conventional encryption technologies such as secure sockets layer (SSL), transport layer security (TLS), virtual private networks (VPNs), Internet Protocol security (IPsec), etc.

The mapping server 625 may store a model that establishes a mapping between monitored biopotential signals with eye-tracking information parameter values. The model may be a machine learning model, a look-up table, etc. In some embodiments described herein, the mapping server 625 may generate, update, maintains, or some combination thereof, of the data associated with the model by a mapping system.

The mapping system may include a means to present visual content with controlled movement to a test user across a population of test users, where the test user wears a test device that is coupled to the head of the test user. The mapping system may include a means to receive information regarding eye movements of the test user in response to the presented visual content from eye-tracking cameras mounted on the test device. The mapping system may include a means to receive, concurrently with the information regarding eye movements of the test user, information regarding biopotential signals from a plurality of electrodes mounted on the test device. Furthermore, the mapping system is configured such that within the plurality of electrodes mounted on the test device, at least some electrodes are in a same configuration as the plurality of electrodes on the device of the user (e.g., headset 100 in FIG. 1A, headset 105 in FIG. 1B, in-ear device 210 in FIG. 2). The mapping system may store the concurrently received information regarding the eye movements and the biopotential signals for the population of test users.

In some embodiments, the information obtained from the population of test users may be used by the mapping system to train machine learning and/or deep learning models, such as regression models, reinforcement models, neural networks, encoder/decoder models such as auto-encoders, etc., to establish the correlation between monitored biopotential signals and eye-tracking movement parameter values.

The mapping system may generate, update, and maintain the model on the mapping server 625. In some embodiments, the model may be maintained as a function that maps the monitored biopotential signals and eye-tracking movement parameter values. In some embodiments, the model may be maintained as a look-up table that maps the monitored biopotential signals and eye-tracking movement parameter values. The mapping server 625 may send the model to the eye-tracking system 420 through the network 620 upon receiving a request from the eye-tracking system 420. In some embodiments, the mapping server 625 may periodically push an updated model to the eye-tracking system 420.

The mapping server 625 may include a database that stores a virtual model describing a plurality of spaces, wherein one location in the virtual model corresponds to a current configuration of a local area of the headset 605. The mapping server 625 receives, from the headset 605 via the network 620, information describing at least a portion of the local area and/or location information for the local area. The user may adjust privacy settings to allow or prevent the headset 605 from transmitting information to the mapping server 625. The mapping server 625 determines, based on the received information and/or location information, a location in the virtual model that is associated with the local area of the headset 605. The mapping server 625 determines (e.g., retrieves) one or more acoustic parameters associated with the local area, based in part on the determined location in the virtual model and any acoustic parameters associated with the determined location. The mapping server 625 may transmit the location of the local area and any values of acoustic parameters associated with the local area to the headset 605

One or more components of system 600 may contain a privacy module that stores one or more privacy settings for user data elements. The user data elements describe the user or the headset 605. For example, the user data elements may describe a physical characteristic of the user, an action performed by the user, a location of the user of the headset 605, a location of the headset 605, an HRTF for the user, etc. Privacy settings (or “access settings”) for a user data element may be stored in any suitable manner, such as, for example, in association with the user data element, in an index on an authorization server, in another suitable manner, or any suitable combination thereof.

A privacy setting for a user data element specifies how the user data element (or particular information associated with the user data element) can be accessed, stored, or otherwise used (e.g., viewed, shared, modified, copied, executed, surfaced, or identified). In some embodiments, the privacy settings for a user data element may specify a “blocked list” of entities that may not access certain information associated with the user data element. The privacy settings associated with the user data element may specify any suitable granularity of permitted access or denial of access. For example, some entities may have permission to see that a specific user data element exists, some entities may have permission to view the content of the specific user data element, and some entities may have permission to modify the specific user data element. The privacy settings may allow the user to allow other entities to access or store user data elements for a finite period of time.

The privacy settings may allow a user to specify one or more geographic locations from which user data elements can be accessed. Access or denial of access to the user data elements may depend on the geographic location of an entity who is attempting to access the user data elements. For example, the user may allow access to a user data element and specify that the user data element is accessible to an entity only while the user is in a particular location. If the user leaves the particular location, the user data element may no longer be accessible to the entity. As another example, the user may specify that a user data element is accessible only to entities within a threshold distance from the user, such as another user of a headset within the same local area as the user. If the user subsequently changes location, the entity with access to the user data element may lose access, while a new group of entities may gain access as they come within the threshold distance of the user.

The system 600 may include one or more authorization/privacy servers for enforcing privacy settings. A request from an entity for a particular user data element may identify the entity associated with the request and the user data element may be sent only to the entity if the authorization server determines that the entity is authorized to access the user data element based on the privacy settings associated with the user data element. If the requesting entity is not authorized to access the user data element, the authorization server may prevent the requested user data element from being retrieved or may prevent the requested user data element from being sent to the entity. Although this disclosure describes enforcing privacy settings in a particular manner, this disclosure contemplates enforcing privacy settings in any suitable manner

### Additional Configuration Information

The foregoing description of the embodiments has been presented for illustration; it is not intended to be exhaustive or to limit the patent rights to the precise forms disclosed. Persons skilled in the relevant art can appreciate that many modifications and variations are possible considering the above disclosure.

Some portions of this description describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. Furthermore, it has also proven convenient at times, to refer to these arrangements of operations as modules, without loss of generality. The described operations and their associated modules may be embodied in software, firmware, hardware, or any combinations thereof.

Any of the steps, operations, or processes described herein may be performed or implemented with one or more hardware or software modules, alone or in combination with other devices. In one embodiment, a software module is implemented with a computer program product comprising a computer-readable medium containing computer program code, which can be executed by a computer processor for performing any or all the steps, operations, or processes described.

Embodiments may also relate to an apparatus for performing the operations herein. This apparatus may be specially constructed for the required purposes, and/or it may comprise a general-purpose computing device selectively activated or reconfigured by a computer program stored in the computer. Such a computer program may be stored in a non-transitory, tangible computer readable storage medium, or any type of media suitable for storing electronic instructions, which may be coupled to a computer system bus. Furthermore, any computing systems referred to in the specification may include a single processor or may be architectures employing multiple processor designs for increased computing capability.

Embodiments may also relate to a product that is produced by a computing process described herein. Such a product may comprise information resulting from a computing process, where the information is stored on a non-transitory, tangible computer readable storage medium and may include any embodiment of a computer program product or other data combination described herein.

Finally, the language used in the specification has been principally selected for readability and instructional purposes, and it may not have been selected to delineate or circumscribe the patent rights. It is therefore intended that the scope of the patent rights be limited not by this detailed description, but rather by any claims that issue on an application based hereon. Accordingly, the disclosure of the embodiments is intended to be illustrative, but not limiting, of the scope of the patent rights, which is set forth in the following claims.

What is claimed is:

1. A method comprising:
monitoring biopotential signals from a plurality of electrodes mounted on a device coupled to a head of a user;
determining eye-tracking information for the user using a machine learning model based on the monitored biopotential signals; and
performing at least one action based in part on the determined eye-tracking information.

2. The method of claim 1, wherein the at least one action comprises selectively emphasizing acoustic content received from one or more acoustic sensors based in part on the determined eye-tracking information to generate enhanced audio content.

3. The method of claim 2, wherein selectively emphasizing acoustic content comprises combining information from the one or more acoustic sensors to emphasize acoustic content associated with a particular region of a local area while deemphasizing acoustic content that is from outside of the particular region, the particular region of the local area based on the determined eye-tracking information.

4. The method of claim 1, wherein performing the at least one action comprises:
determining an occurrence of one or more ocular events based on the determined eye-tracking information, wherein the one or more ocular events comprise at least one of: ocular fixation, ocular saccades, ocular movement speed, ocular movement direction, and ocular blink; and
providing information associated with the determined one or more ocular events to a display system on the device, wherein the display system adjusts the display of visual content presented to the user based on the information associated with the determined one or more ocular events.

5. The method of claim 1, further comprising:
concurrent to monitoring the biopotential signals from the plurality of electrodes, receiving information regarding eye movements of the user from one or more eye-tracking cameras mounted on the device; and
combining the information regarding eye movements of the user with the determined eye-tracking information based on the monitored biopotential signals to generate improved eye-tracking information.

6. The method of claim 5, wherein combining the information regarding eye movements of the user with the determined eye-tracking information based on the monitored biopotential signals to generate the improved eye-tracking information comprises:
receiving the information regarding eye movements of the user from the one or more eye-tracking cameras mounted on the device at a first sampling frequency;
monitoring the biopotential signals from the plurality of electrodes at a second sampling frequency, wherein the second sampling frequency is greater than the first sampling frequency; and
using information from the monitored biopotential signals to compensate for missing information in the received information regarding eye movements of the user.

7. The method of claim 1, further comprising:
concurrent to monitoring the biopotential signals from the plurality of electrodes, receiving information regarding eye movements of the user from one or more eye-tracking cameras mounted on the device;
comparing the information regarding eye movements of the user with the determined eye-tracking information based on the monitored biopotential signals;
determining, based on the comparing, that the monitored biopotential signals from the plurality of electrodes exhibit signal drift; and
correcting the determined signal drift in the monitored biopotential signals using one or more signal filters.

8. The method of claim 1, wherein the machine learning model is previously generated, the generating comprising:
for each of a plurality of test users:
presenting visual content with controlled movement to a test user wearing a test device coupled to a head of the test user;
receiving information regarding eye movements of the test user in response to the presented visual content from eye-tracking cameras mounted on the test device;
concurrent to receiving the information regarding eye movements of the test user, receiving information regarding biopotential signals from a plurality of electrodes mounted on the test device, the plurality of electrodes having at least some electrodes in a same configuration as the plurality of electrodes on the device; and
storing the concurrently received information regarding the eye movements and the biopotential signals; and
training the machine learning model based on the stored concurrently received information regarding the eye movements and the biopotential signals.

9. A wearable device assembly comprising:
a headset comprising:
a display assembly;
an audio system; and
an eye-tracking system configured to:
receive biopotential signals from a plurality of electrodes that are configured to monitor biopotential signals generated within a head of a user in response to eye movements of the user,
determine eye-tracking information for the user using a trained machine learning model based on the monitored biopotential signals; and
wherein at least one of the display assembly or the audio system are configured to perform at least one action based in part on the determined eye-tracking information.

10. The wearable device assembly of claim 9, wherein electrodes in the plurality of electrodes are soft, flat, and foldable electrodes that comprise at least one of:
a plurality of silver chloride electrodes;
a plurality of iridium oxide electrodes on a titanium substrate; and
a plurality of gold-plated electrodes.

11. The wearable device assembly of claim 9, wherein the plurality of electrodes is further configured to:
include electrodes that are spatially distributed on end pieces of a frame of the headset; and
include a ground electrode that is mounted on a front part of the frame of the headset.

12. The wearable device assembly of claim 11, wherein, when the electrodes are spatially distributed on the end pieces of the frame of the headset, a signal to noise ratio of the monitored biopotential signals is above a prespecified target threshold.

13. The wearable device assembly of claim 9, wherein the plurality of electrodes includes electrodes that are mounted on the headset and are positioned to be in contact with a forehead region above an eye of the user, and electrodes that are mounted on the headset and positioned to be in contact with a facial cheek region below the eye.

14. The wearable device assembly of claim 9, further comprising an in-ear device assembly, the in-ear device assembly comprising one or more in-ear devices.

15. The wearable device assembly of claim 14, wherein, the plurality of electrodes is further configured to:
include electrodes that are spatially distributed on an outer surface of an in-ear device; and
include electrodes that are located on the outer surface of the in-ear device and that touch an ear canal region and a conchal bowl region of the user.

16. The wearable device assembly of claim 9, wherein the eye tracking system is further configured to perform the at least one action comprising:
determining an occurrence of one or more ocular events based on the determined eye-tracking information, wherein the one or more ocular events comprise at least one of: ocular saccade, ocular fixation, ocular blink, and ocular movement in a particular direction at a particular speed; and
providing eye-tracking information associated with the determined one or more ocular events to at least one of: the display assembly, an optics block of the headset, and the audio system.

17. The wearable device assembly of claim 16, wherein at least one of: the display system and the optics block are further configured to adjust a display of visual content presented to the user based on the provided information associated with the determined one or more ocular events from the controller.

18. The wearable device assembly of claim 16, wherein the audio system is further configured to selectively emphasize acoustic content to generate enhanced audio content, the selectively emphasizing comprising emphasizing acoustic content associated with a particular region of a local area while deemphasizing acoustic content that is from outside of the particular region, the particular region of the local area based on the determined eye-tracking information.

19. The wearable device assembly of claim 9, further comprising:
concurrent to receiving the biopotential signals from the plurality of electrodes, receiving information regarding eye movements from one or more eye-tracking cameras mounted on the headset; and
combine the information regarding eye movements of the user with the determined eye-tracking information based on the monitored biopotential signals to generate improved eye-tracking information.

20. The wearable device assembly of claim 9, further comprising:
concurrent to receiving the biopotential signals from the plurality of electrodes, receiving information regarding eye movements of the user from one or more eye-tracking cameras mounted on the headset;
compare the information regarding eye movements of the user with the determined eye-tracking information based on the monitored biopotential signals;
determine, based on the comparing, that the monitored biopotential signals from the plurality of electrodes include signal drift; and
correct the determined signal drift in the monitored biopotential signals using one or more signal filters.
